(19) United States
(12) Patent Application Publication
(10) Pub. No.: US 2024/0308970 A1
(43) Pub. Date: Sep. 19, 2024

(54) METHOD FOR PRODUCING ALKYLENE OXIDES

(71) Applicant: Covestro Deutschland AG, Leverkusen (DE)

(72) Inventors: Matthias Leven, Köln (DE); Norah Heinz, Leverkusen (DE); Ulrich Liman, Langenfeld (DE); Jens Langanke, Mechernich (DE); Torsten Heinemann, Leichlingen (DE)

(21) Appl. No.: 18/571,783

(22) PCT Filed: Jun. 28, 2022
(86) PCT No.: PCT/EP2022/067640

§ 371 Date: Dec. 19, 2023

(30) Foreign Application Priority Data
Jun. 30, 2021 (EP) 21182701.9

(51) Int. Cl. C07D 301/10

(52) U.S. Cl. CPC C07D 301/10

(57) ABSTRACT

The invention relates to a method for producing alkylene oxides by reacting an alkene with an arene oxide, pyridine-N-oxide, and/or pyrimidine-N-oxide, preferably an arene oxide and/or pyridine-N-oxide, in the presence of a catalyst in a first reactor, wherein the catalyst comprises a metal and/or a metal salt, and the metal is copper, silver, and/or gold. The metal salt comprises chrome, iron, cobalt, and/or copper cation(s), and the reaction is carried out in the absence of oxygen or an oxygen-containing gas mixture.

The invention provides a process for producing an alkylene oxide by reacting an alkene with an arene oxide, pyridine N-oxide and/or pyrimidine N-oxide, preferably with an arene oxide and/or pyridine N-oxide, preferably in the presence of a catalyst in a first reactor, where the catalyst comprises a metal and/or a metal salt, where the metal is copper, silver and/or gold, where the metal salt comprises chromium, iron, cobalt and/or copper cation(s), and where the reaction is effected in the absence of oxygen or an oxygen-containing gas mixture.

The prior art has described numerous processes for preparing alkylene oxides, specifically propylene oxide, where the chlorohydrin process in particular is of industrial significance, in which propene is reacted with hypochlorous acid or chlorine and water an isomer mixture of 1-chloro-2-propanol and 2-chloro-1-propanol, which is subsequently reacted with milk of lime to give propylene oxide and calcium chloride. However, the formation of a CaCl₂) salt burden results in high wastewater contamination or an additional recycling step and incorporation into a chlor-alkali operation. This technology is rated as disadvantageous in terms of carbon footprint and greenhouse gas emissions (Reduction of GHG Emissions in Propylene Oxide Production, Approved VCS Methodology VM0023 Version 1.0, 9 Sep. 2013 Sectoral Scope 5, South Pole Carbon Asset Management Ltd.).

Additionally of relevance is the preparation of propylene oxide by means of coproduct-based methods (e.g. oxirane method), in which ethylbenzene or isobutane is converted in a 1st stage in the presence of oxygen to the respective hydroperoxides, which can then be reacted with propene to give propylene oxide and 1-phenylethanol or tert-butanol as further coproducts. These coproducts can subsequently be converted further to styrene and isobutene or isobutane. However, aside from the propylene oxide, a coproduct is likewise formed, which additionally has to be removed and processed further in further plants. The coproduct-based methods always require a sales market for the coproduct, and make the economics of such processes complex and prone to adverse effects. Thus, as well the technically demanding processes, there are also obvious market-economic risks, and so a technological and economic assessment of these processes does not show a very advantageous overall picture. This is especially because the demand/sales situation for both products, PO and the coproduct, are generally different regionally. In other words, long-range logistics are needed, which entail direct costs and further disadvantages, such as CO2/GHG emissions. The industrial MTBE process requires incorporation into a refinery operation in order to make a maximum economic contribution.

Also of relevance on an industrial scale is what is called the HPPO process, in which propylene is reacted with hydrogen peroxide to give propylene oxide and water. What is advantageous over the aforementioned industrial scale preparation processes is that no coproduct or salt burden arises in the product. However, hydrogen peroxide has to be produced in an upstream, technically complex catalytic process. The process is considered to be technically demanding and is generally operated only at integrated sites with other H2O2 consumers.

The direct oxidation of propene to propylene oxide is additionally considered to be technically immature, and, according to the present state of the art, there is no direct oxidation process which is performable economically on an industrial scale. Particularly the reaction regime and especially the control of temperature in gas phase and salt melt processes are an unsolved challenge, even though a high propene conversion in conjunction with a high PO selectivity is crucial for an efficient industrial process. In the case of direct oxidation, a number of by-products and conversion products, for example methanol, acetaldehyde, carbon dioxide, ethylene and formaldehyde, occur in substantial amounts (cf. D. Kahlich et al., Dow Deutschland in Ullmann's Encyclopedia of Industrial Chemistry, chapter entitled “Propylene Oxide”, Wiley VCH, 2012). For example, current prior art documents that may be cited in respect of the aerobic oxidation of alkenes such as propene are US2018208569A1 and US2020346193A1. The respective epoxy selectivities are only 54.5% and 60%, with respective conversions of 8.6% and 5%.

Arene oxides are key compounds in the oxidative metabolism of aromatic compounds. They can be prepared in high yields by enzymatic synthesis methods. Scientific publications that describe these processes in detail for the purposes of synthesis are, for example, “Enzymatic and chemoenzymatic synthesis of arene trans-dihydrodiols” in Journal of Molecular Catalysis B: Enzymatic, Volumes 19-20, 2, 2002, 31-42 or “Chemical Equivalent of Arene Monooxygenases: Dearomative Synthesis of Arene Oxides and Oxepines” by Zohaib Siddiqi, William C. Wertjes, and David Sarlah, J. Am. Chem. Soc. 2020, 142, 22, 10125-10131. But there are also other synthesis methods that are suitable production methods, such as direct epoxidation, preparation from trans-1,2-glycols, preparation from cis-1,2-glycols, preparation from bromohydrins, preparation from vicinal dihalogen compounds of epoxidized protoaromatics, ring closure of seco derivatives, electrochemical oxidation, reaction of annulenes, reaction of arene photooxides and/or -peroxides, reaction of ozonide compounds, preparation from oxygen heterocycles, and intramolecular oxygen transfer reaction (intramolecular oxygen trapping) (cf., for example, G. S. Shirwaiker et al., Advances in Heterocyclic Chemistry, Vol. 37, 1984, 67-165).

Arene oxides are obtained by the oxidation of aromatic and polycyclic aromatic compounds. Typically, hypochlorites are used here as oxidizing agents in combination with phase transfer catalysts (cf., for example, JP61109784). The reactions are conducted either in neat form or in solvents; chlorinated and nitrated hydrocarbons in particular have been found to be useful here. Monitoring of pH may be advantageous in the synthesis.

Arene oxides of various halogenated benzenes are also commercially available.

The selective oxidation of ternary nitrogen compounds has been very well described. There have been descriptions, for example, of processes using hydrogen peroxide, and the use of methyltrioxorhenium(VII) (J. Org. Chem., 1995, 60, 1326), magnesium porphyrins (Synthesis, 1997, 1387), flavins (1980, 102, 6498), TS-1 and other zeolites (Catal. Lett., 2001, 72, 233), molecular sieves (Chem. Commun., 2000, 1577), polyoxometallates (J. Mol. Catalysis A: Chem. 252, 219-225, 2006; Green Chem., 2011, 13, 1486-1489) or tungsten-substituted Mg—Al layer hydroxides (Chem. Commun., 2001, 1736) as catalysts. In addition, the person skilled in the art is aware of other synthesis methods using different inorganic and organic oxidizing agents, for example organic acids, dioxiranes, peroxomonosulfuric acid (Caro acid), peroxomonophosphoric acid, etc. (cf., inter alia, Michael B. Smith, March's Advanced Organic Chemistry, 7th Edition, Wiley, 2012).

N-oxides of aromatic N-heterocyclic compounds can be obtained as catalyst by oxidation with the aid of oxygen and ruthenium(III) chloride as catalyst (S. L. Jain et al. Chem. Commun. 2002, 1040-1041). At 20° C. and 1 atm O2, it is possible to obtain N-oxides of pyridine, 2-, 3- and 4-picolines, substituted pyridines, quinoline and isoquinoline in yields of up to 95%. The use of particular aprotic solvents and/or additional P ligands can have a positive effect on the kinetics of the method.

CN108623519 describes the oxidation of pyridine with oxygen in isopropanol in the presence of a titanium-containing zeolite catalyst. This is a technically simple process, and high conversions and high yields are reported.

The use of N-oxides as oxidizing agents is known (cf., for example, Org. Synth. Coll. Vol., 1988, 6, 342; Chem. Rev., 1980, 80, 187; Org. Proc. Res. Dev., 1997, 1, 425; Synthesis, 1994, 639; Chem. Ber., 1961, 94, 1360; Bull. Chem. Soc. Jpn., 1986, 59, 3287; Tetrahedron Lett., 1990, 31, 4825; J. Chem. Soc., Chem. Commun., 1987, 1625). N-oxides are found to be efficient transferers of atomic oxygen, with recovery of the original nitrogen compound. This can then be recycled in a simple manner or oxidatively converted in situ directly to the corresponding N-oxide. When N-oxidic oxidants are used, there is generally no overoxidation or formation of by-products and/or further conversion products, especially when selective catalyst systems are used.

The selective catalytic conversion of unsaturated compounds to epoxides using N-oxides has been well described in the literature (RSC Adv., 2016, 6, 88189-88215). The epoxidation of nonaromatic linear α-olefins with N-oxides has to date been accomplished solely using costly ruthenium-based porphyrin catalysts and very long reaction times: For example, the epoxidation of 1-octene using a ruthenium-porphyrin catalyst (empirical formula C₈₅H₇₆ON₄Ru) and 2,6-dichloropyridine N-oxide in benzene as solvent at 125° C. in a Schlenk tube after 48 hours achieved a conversion of only 6% and an epoxide yield of 5% (J. Chem. Soc., Perkin Trans. 1, 1997). In a further example, the epoxidation of 1-octene using an MCM-41-supported ruthenium-porphyrin catalyst and 2,6-dichloropyridine N-oxide in dichloromethane as solvent at 40° C. after 24 hours achieved a conversion of 80% and an epoxide yield of 74% (J. Org. Chem., 1998, 63, 7364-7369). In a further example, the epoxidation of 1-octene using a PEGylated ruthenium-porphyrin catalyst and 2,6-dichloropyridine N-oxide in dichloromethane as solvent at 50° C. after 24 hours achieved a conversion of 82% and an epoxide yield of 81% (Chem.-Eur. J., 2006, 12, 3020-3031). A theoretical study (RSC Adv., 2016, 6, 88189-88215) suggests the use of Ru(meso-tetrakis(2,6-dichlorophenyl)porphyrin) as catalyst in combination with dimethylpyridine N-oxide for epoxidation of propene to propylene oxide, without specifying reaction conditions.

Overall, the low reaction temperatures in combination with the long reaction times show that these Ru-based catalysts have only low added value for industrial use in the epoxidation of linear terminal alkenes. It is possible to achieve usable selectivities only when very low reaction temperatures are chosen. In addition, solvents that are not preferable for use on an industrial scale are used. The catalysts used in the prior art contain Ru in the (+II) and (+IV) oxidation states.

It was therefore an object of the present invention to provide a catalyst system for the direct production of alkylene oxides (epoxides), preferably of propylene oxide, which, in the oxidative conversion of alkenes in the absence of oxygen or an oxygen-containing gas mixture, has a with improved epoxide conversion compared to systems known from the prior art and improved product selectivity via reduction in the formation of unwanted by-products, such as allylic compounds and the further conversion products thereof, and the avoidance of formation of nonrecyclable coproducts. This improved catalyst activity and selectivity should be reflected by an activation energy E_{A} of up to 24.0 kcal/mol, preferably of 10.0 kcal/mol to 21.0 kcal/mol, for the transfer of oxygen to the alkene, preferably the propene, where side reactions or further conversion reactions have higher activation energies than the preferred alkoxylation reaction. Higher activation energies of more than 24.0 kcal/mol would require higher reaction temperatures that favor the unwanted side reactions already discussed, but also others. Moreover, the activation energies of side reactions or further conversion reactions should be not less than 10.0 kcal/mol, in order that control of selectivity via reaction temperature at greater than 20° C. is possible.

It has been found that, surprisingly, the object of the invention is achieved by a process for producing an alkylene oxide by reacting an alkene with an arene oxide, pyridine N-oxide and/or pyrimidine N-oxide, preferably with an arene oxide and/or pyridine N-oxide, in the presence of a catalyst (A) in a first reactor, wherein the catalyst (A) comprises a metal (A-1) and/or a metal salt (A-2), wherein the metal (A-1) is copper, silver and/or gold, wherein the metal salt (A-2) comprises chromium (Cr), iron (Fe), cobalt (Co) and/or copper (Cu) cation(s), and wherein the reaction is effected in the absence of oxygen or an oxygen-containing gas mixture.

The embodiments that follow may be combined as desired, unless the opposite is apparent from the technical context and common art knowledge.

The alkylene oxide (epoxide) of the invention may be an alkylene oxide having 2-45 carbon atoms. In one embodiment of the process of the invention, the alkylene oxide is selected from at least one compound from the group consisting of ethylene oxide, propylene oxide, 1,2-butylene oxide, 2,3-butylene oxide, 2-methyl-1,2-propylene oxide (isobutene oxide), 1,2-pentylene oxide, 2,3-pentylene oxide, 2-methyl-1,2-butylene oxide, 3-methyl-1,2-butylene oxide, alkylene oxides of C6-C22 α-olefins, such as 1,2-hexylene oxide, 2,3-hexylene oxide, 3,4-hexylene oxide, 2-methyl-1,2-pentylene oxide, 4-methyl-1,2-pentylene oxide, 2-ethyl-1,2-butylene oxide, 1,2-heptylene oxide, 1,2-octylene oxide, 1,2-nonylene oxide, 1,2-decylene oxide, 1,2-undecylene oxide, 1,2-dodecylene oxide, 4-methyl-1,2-pentylene oxide, cyclopentylene oxide, cyclohexylene oxide, cycloheptylene oxide, cyclooctylene oxide, styrene oxide, methylstyrene oxide, pinene oxide, allyl glycidyl ether, vinylcyclohexylene oxide, cyclooctadiene monoepoxide, cyclododecatriene monoepoxide, butadiene monoepoxide, isoprene monoepoxide, limonene oxide, 1,4-divinylbenzene monoepoxide, 1,3-divinylbenzene monoepoxide, glycidyl acrylate and glycidyl methacrylate, mono- or polyepoxidized fats as mono-, di- and triglycerides, epoxidized fatty acids, C1-C24 esters of epoxidized fatty acids, epichlorohydrin, glycidol, and derivatives of glycidol, for example glycidyl ethers of C1-C22 alkanols and glycidyl esters of C1-C22 alkanecarboxylic acids. Examples of derivatives of glycidol are phenyl glycidyl ether, cresyl glycidyl ether, methyl glycidyl ether, ethyl glycidyl ether and 2-ethylhexyl glycidyl ether.

In a preferred embodiment of the process, the alkylene oxide is ethylene oxide, propylene oxide, 1,2-butylene oxide, 1,2-pentylene oxide, 1,2-hexylene oxide, 1,2-heptylene oxide and/or 1,2-octylene oxide. In a particularly preferred embodiment of the process, the alkylene oxide is ethylene oxide and/or propylene oxide. In a very particularly preferred embodiment of the process, the alkylene oxide is propylene oxide.

In one embodiment of the process of the invention, the alkene is one or more compound(s) and is selected from the group consisting of ethene, propene, butene, 1-octene, butadiene, butane-1,4-diol diallyl ether, allyl chloride, allyl alcohol, styrene, cyclopentene, cyclohexene, phenyl allyl ether, diallyl ether, n-butyl allyl ether, tert-butyl allyl ether, bisphenol A diallyl ether, resorcinol diallyl ether, triphenylolmethane triallyl ether, cyclohexane-1,2-dicarboxylic acid bis(allyl ester), isocyanuric acid tris(prop-2,3-ene) ester and mixtures of these alkenes, preferably ethene, propene and allyl chloride, and more preferably propene.

In one embodiment of the process of the invention, the arene oxide is one or more compounds of formula (I), (II), (III) and/or (IV):

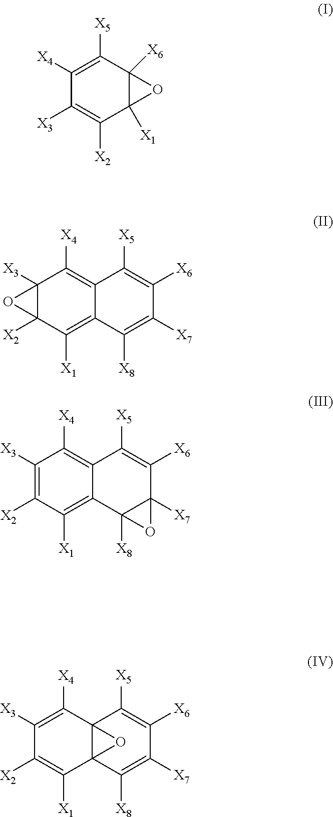

with
X₁ to X₈ independently selected from the group of F, Cl, Br, CN, —CO—CF₃, —CO—C(CH₃)₃, —CO—CH₂C(CH₃)₃, —CO—C₆H₅, —CO—OC(CH₃)₃, —CO—OCH₂C(CH₃)₃ and H, preferably F, Cl, more preferably Cl.

In a preferred embodiment of the process of the invention, the arene oxide is one or more compounds and is selected from the group consisting of hexafluorobenzene oxide, hexachlorobenzene oxide, 1-bromo-2,3,4-trifluorobenzene oxide, pentafluorobenzene oxide, 1,3,5-trichloro-2,4,6-trifluorobenzene oxide, 1,3,5-trifluorobenzene oxide, 1,2-dibromo-3,5-difluorobenzene oxide, 1,2,4,5-tetrafluorobenzene oxide, bromopentafluorobenzene oxide, 1,3,5-trichlorobenzene oxide, 1-bromo-3,5-dichlorobenzene oxide, orthodichlorobenzene oxide, 1,2,4,5-tetrachlorobenzene oxide, 1,2,3-trichlorobenzene oxide and 1,5-dichloro-2-fluorobenzene oxide, preferably hexafluorobenzene oxide and hexachlorobenzene oxide.

In one embodiment of the process of the invention, the arene oxide is obtainable by reacting a first aromatic compound with oxygen or an oxygen-containing gas mixture in the presence of a catalyst (C).

In one embodiment of the process of the invention, the arene oxide is prepared by reacting a first aromatic compound with oxygen or an oxygen-containing gas mixture in the presence of a catalyst (C).

The oxygen-containing gas mixture, as well as oxygen, also comprises diluent, carrier or inert gases such as hydrocarbons, noble gases, CO, CO2 and/or N2.

In one embodiment of the process of the invention, further additives are added to the oxygen or an oxygen-containing gas mixture, such as water, CO, N-containing compounds, for example hydrazine, ammonia (NH3), methylamine (MeNH₂), NOx, PH₃, SO₂ and/or SO₃, in amounts of 10 ppm to 500 ppm, preferably of 30 ppm to 300 ppm and more preferably of 50 ppm to 200 ppm. In addition, CO2 may be added in a proportion of 0.01% by volume to 50% by volume, preferably of 0.1% by volume to 20% by volume, and more preferably of 1% by volume to 10% by volume. It is also possible to add organic halides, such as ethylene dichloride, ethyl chloride, vinyl chloride, methyl chloride and/or methylene chloride, in amounts of 10 ppm to 500 ppm, preferably of 50 ppm to 400 ppm, and more preferably of 100 ppm to 300 ppm.

In one embodiment of the process of the invention, the first aromatic compound has a boiling temperature of 50° C. to 350° C. at 1 bara.

In one embodiment of the process of the invention, the first aromatic compound is one or more compound(s) and is selected from the group consisting of hexafluorobenzene, hexachlorobenzene, 1-bromo-2,3,4-trifluorobenzene, pentafluorobenzene, 1,3,5-trichloro-2,4,6-trifluorobenzene, 1,3,5-trifluorobenzene, 1,2-dibromo-3,5-difluorobenzene, 1,2,4,5-tetrafluorobenzene, bromopentafluorobenzene, 1,3,5-trichlorobenzene, 1-bromo-3,5-dichlorobenzene, orthodichlorobenzene, 1,2,4,5-tetrachlorobenzene, 1,2,3-trichlorobenzene and 1,5-dichloro-2-fluorobenzene, preferably hexafluorobenzene and hexachlorobenzene.

In one embodiment of the process of the invention, the catalyst (C) is one or more compound(s) and is selected from the group consisting of silver, silver supported on magnesium silicate, and cytochrome P450 enzyme.

In one embodiment of the process of the invention, the molar ratio of oxygen to the first aromatic compound is from 1:500 to 1:1, preferably from 1:100 to 1:1.

In one embodiment of the process of the invention, the catalyst (C) is used in a calculated amount of 10 ppm to 15%, preferably of 100 ppm to 5% and more preferably of 100 ppm to 2%, based on the amount of the first aromatic compound used.

In one embodiment of the process of the invention, the arene oxide is produced at a temperature of 20° C. to 250° C., preferably of 50° C. to 220° C. and more preferably of 100° C. to 200° C.

In one embodiment of the process of the invention, the arene oxide is produced at a pressure of 1 bara to 200 bara, preferably of 1 bara to 100 bara and more preferably of 1 bara to 60 bara.

In one embodiment of the process of the invention, the arene oxide is produced within a period of 6 min to 48 h, preferably of 6 min to 24 h and more preferably of 6 min to 3 h

In one embodiment of the process of the invention, the pyridine N-oxide is one or more compounds of formula (V):

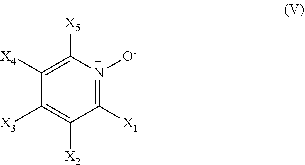

with
X₁ to X₅ independently selected from the group of F, Cl, Br, CN, —CO—CF₃, —CO—C(CH₃)₃, —CO—CH₂C(CH₃)₃, —CO—C₆H₅, —CO—OC(CH₃)₃, —CO—OCH₂C(CH₃)₃ and H, preferably F, Cl, more preferably Cl.

In one embodiment of the process of the invention, the pyridine N-oxide is one or more compound(s) and is selected from the group consisting of pentafluoropyridine N-oxide, 2-bromo-3,5-dichloropyridine N-oxide, 3-chloropyridine N-oxide, 3,6-dichloropyridine N-oxide, 3,5-dichloropyridine N-oxide, 3-chloro-2,5,6-trifluoropyridine N-oxide, 3-chloro-2,4,5,6-tetrafluoropyridine 1-N-oxide, 3-chloro-2,4,5,6-tetrafluoropyridine 3-N-oxide, preferably pentafluoropyridine N-oxide.

In one embodiment of the process of the invention, the pyridine N-oxide is obtainable by reacting a second aromatic compound with oxygen or an oxygen-containing gas mixture in the presence of a catalyst (D).

In one embodiment of the process of the invention, the pyridine N-oxide is prepared by reacting a second aromatic compound with oxygen or an oxygen-containing gas mixture in the presence of a catalyst (D).

In one embodiment of the process of the invention, the second aromatic compound has a boiling temperature of 50° C. to 350° C. at 1 bara.

In one embodiment of the process of the invention, the second aromatic compound is one or more compound(s) and is selected from the group consisting of pentafluoropyridine N-oxide, 2-bromo-3,5-dichloropyridine, 3-chloropyridine, 3,6-dichloropyridine, 3,5-dichloropyridine, 3-chloro-2,5,6-trifluoropyridine, 3-chloro-2,4,5,6-tetrafluoropyridine, 3-chloro-2,4,5,6-tetrafluoropyridine, preferably pentafluoropyridine.

In one embodiment of the process of the invention, the catalyst (D) is one or more compound(s) and is selected from the group consisting of ruthenium trichloride, titanium-containing zeolites and silver.

In one embodiment of the process of the invention, the catalyst (D) is applied to a catalyst support, and the catalyst support is one or more compounds and is selected from the group consisting of magnesium silicate, carbon, silica gel, aluminum oxide, titanium dioxide and cation exchange resin.

In one embodiment of the process of the invention, the molar ratio of oxygen to the second aromatic compound is from 1:500 to 1:1, preferably from 1:100 to 1:1.

In one embodiment of the process of the invention, the catalyst (D) is used in a calculated amount of 10 ppm to 15%, preferably of 100 ppm to 5% and more preferably of 100 ppm to 2%, based on the amount of the second aromatic compound used.

In one embodiment of the process of the invention, the pyridine N-oxide is produced at a temperature of 0° C. to 250° C., preferably of 10° C. to 220° C. and more preferably of 20° C. to 150° C.

In one embodiment of the process of the invention, the pyridine N-oxide is produced at a pressure of 1 bara to 200 bara, preferably of 1 bara to 100 bara and more preferably of 1 bara to 60 bara.

In one embodiment of the process of the invention, the pyridine N-oxide is produced within a period of 6 min to 48 h, preferably of 6 min to 24 h and more preferably of 6 min to 3 h

In one embodiment of the process of the invention, the pyrimidine N-oxide is one or more compounds of formula (VI) and/or (VII):

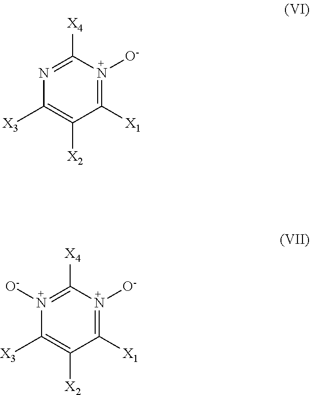

with
X₁ to X₄ independently selected from the group of F, Cl, Br, CN, —CO—CF₃, —CO—C(CH₃)₃, —CO—CH₂C(CH₃)₃, —CO—C₆H₅, —CO—OC(CH₃)₃, —CO—OCH₂C(CH₃)₃ and H, preferably F, Cl, more preferably Cl.

In one embodiment of the process of the invention, the pyrimidine N-oxide is one or more compounds and is selected from the group consisting of 2-chloropyrimidine N-oxide, 2,4-dichloro-6-methylpyrimidine 1-N-oxide, 2,4-dichloro-6-methylpyrimidine 3-N-oxide, 2,5-dichloropyrimidine 1-N-oxide, 2,5-dichloropyrimidine 2-N-oxide.

In one embodiment of the process of the invention, the pyrimidine N-oxide is obtainable by reacting a third aromatic compound with oxygen or an oxygen-containing gas mixture in the presence of a catalyst (E).

In one embodiment of the process of the invention, the pyrimidine N-oxide is prepared by reacting a third aromatic compound with oxygen or an oxygen-containing gas mixture in the presence of a catalyst (E).

In one embodiment of the process of the invention, the third aromatic compound has a boiling temperature of 50° C. to 350° C. at 1 bara.

In one embodiment of the process of the invention, the third aromatic compound is one or more compound(s) and is selected from the group consisting of 2-chloropyrimidine, 2,4-dichloro-6-methylpyrimidine, 2,4-dichloro-6-methylpyrimidine, 2,5-dichloropyrimidine, preferably 2,4-dichloro-6-methylpyrimidine.

In one embodiment of the process of the invention, the catalyst (E) is one or more compound(s) and is selected from the group consisting of ruthenium trichloride, titanium-containing zeolites and silver.

In one embodiment of the process of the invention, the molar ratio of oxygen to the third aromatic compound is from 1:500 to 1:1, preferably from 1:100 to 1:1.

In one embodiment of the process of the invention, the catalyst (E) is used in a calculated amount of ppm to 15%, preferably of 100 ppm to 5% and more preferably of 100 ppm to 2%, based on the amount of the third aromatic compound used.

In one embodiment of the process of the invention, the pyrimidine N-oxide is produced at a temperature of 0° C. to 250° C., preferably of 10° C. to 220° C. and more preferably of 20° C. to 150° C.

In one embodiment of the process of the invention, the pyrimidine N-oxide is produced at a pressure of 1 bara to 200 bara, preferably of 1 bara to 100 bara and more preferably of 1 bara to 60 bara.

In one embodiment of the process of the invention, the pyrimidine N-oxide is produced within a period of 6 min to 48 h, preferably of 6 min to 24 h and more preferably of 6 min to 3 h

In one embodiment of the process of the invention, the alkylene oxide is prepared in the presence of a catalyst (A), where catalyst (A) comprises a metal (A-1) and/or a metal salt (A-2).

In one embodiment of the process of the invention, the metal (A-1) is copper (Cu), silver (Ag) and/or gold (Au), preferably silver (Ag).

In one embodiment of the process of the invention, the metal cation of the metal salt (A-2) has an oxidation state of (+I), (+II); (+III) or (+IV), preferably of (+II); (+III) or (+IV).

In one embodiment of the process of the invention, the metal salt (A-2) is a nitrate, halide, tetrafluoroborate, paratoluenesulfonate, methanesulfonate and/or triflate, preferably a chloride.

In one embodiment of the process of the invention, the metal salt (A-2) is one or more compound(s) and is selected from the group consisting of Cr₂(SO₄)₃, KCr(SO₄)₂, Cr(NO₃)₃, CrF₃, CrCl₃, FeCl₃, FeBr₃, iron triflate, FePO₄, Fe₂(SO₄)₃, Fe(NO₃)₃, FeF₃, iron paratoluenesulfonate, CoCl₂, CoBr₂, Co(NO₃)₂, CoBr₂, CoSO₄, CoF₂, Co(BF₄)₂, Co₃(PO₄)₂, CuCl₂, CuSO₄, (CF₃SO₃)₂Cu, CuF₂, Cu(NO₃)₂, copper(II) pyrophosphate, CuCl, CuI and CuBr, preferably CrCl₃, FeCl₃, CoCl₂ and CuCl₂. The metal salts here may also be in the form of hydrates.

In one embodiment of the process of the invention, the catalyst (A) is used in a calculated amount of 10 ppm to 15%, preferably of 100 ppm to 5% and more preferably of 100 ppm to 2%, based on the amount of the all components used.

In one embodiment of the process of the invention, the catalyst (A) is applied to a catalyst support (B) to form a supported catalyst (A′).

In one embodiment of the process of the invention, the catalyst support (B) is a metal oxide, an alkaline earth metal carbonate, a silicate, a silicon carbide, a silicon oxycarbide, a silicon nitride, a silicon oxynitride and/or a silicon dioxide.

In a preferred embodiment of the process of the invention, the catalyst support (B) is one or more compound(s) and is selected from the group consisting of aluminum oxide aluminum dioxide, silica, titanium dioxide, zirconium dioxide, aluminium carbonate, phyllosilicate, such as talc, kaolinite and pyrophyllite, and titanium dioxide.

In one embodiment of the process of the invention, catalyst (A) is applied to the catalyst support (B) in a calculated proportion by mass of 1.0% by weight to 30.0% by weight.

In one embodiment of the process of the invention, catalyst (A) is applied to the catalyst support (B) by the wet infiltration method or the incipient wetness method.

In one embodiment of the process of the invention, the molar ratio of the alkene to the arene oxide is from 1:0.01 to 10:1, preferably from 1:0.1 to 1:1.

In one embodiment of the process of the invention, the alkylene oxide is produced in the presence of a solvent.

In one embodiment of the process of the invention, the solvent is one or more compound(s) and is selected from the group consisting of CO2, water, perfluoromethyldecalin, perfluorodecalin, perfluoroperhydrophenanthrene, perfluoro(butyltetrahydrofuran), tetrahydrofuran, 2-methyl-THF, acetic acid, acetonitrile, dimethyl sulfoxide, sulfolane, acetone, ethyl methyl ketone, dimethylformamide, dichloromethane, chloroform, tetrachloromethane, N-methyl-2-pyrrolidinone, methyl t-butyl ether (MTBE), dimethyl sulfoxide (DMSO), hexamethylphosphoramide, dichlorobenzene, 1,2-dichloroethylene, 1,1,1,3,3,3-hexafluoroisopropanol, perfluoro-tert-butyl alcohol, 1,1,2,3,3-pentafluoropropane, 1-bromo-2-chloro-1,1,2-trifluoroethane, 1,2-dichloro-1,1,2,3,3,3-hexafluoropropane, ethylene glycol, glycerol, and phenol.

In one embodiment of the process of the invention, the production is effected at a temperature of 20° C. to 200° C., preferably of 50° C. to 160° C. and more preferably of 100° C. to 150° C.

In one embodiment of the process of the invention, the production is effected at a pressure of 1 bara to 200 bara, preferably of 1 bara to 35 bara and more preferably of 1 bara to 28 bara.

In one embodiment of the process of the invention, the production is effected within a period of 6 min to 48 h, preferably of 6 min to 24 h and more preferably of 6 min to 3 h.

In one embodiment of the process of the invention, the molar ratio of the alkene to the oxygen is from 1:100 to 100:1, preferably from 1:30 to 30:1.

In one embodiment of the process of the invention, the production is effected in the absence of a solvent.

What is meant here in accordance with the invention by a production process in the absence of a solvent is that solvent residues, for example as a result of the production of the feedstocks, may be present at up to 10% by volume, preferably up to 5% by volume and more preferably up to 2% by volume, based on the amount of propene used.

In one embodiment of the process of the invention, production is effected using the supported catalyst (A′) of the invention.

In one embodiment of the process of the invention, the production is effected at a temperature of 20° C. to 500° C., preferably of 50° C. to 400° C. and more preferably of 50° C. to 250° C.

In one embodiment of the process of the invention, the production is effected at a pressure of 1 bara to 200 bara, preferably of 2 bara to 100 bara and more preferably of 1 bara to 50 bara.

In one embodiment of the process of the invention, the production is effected with a space velocity of 100 h-1 to 10 000 h-1, preferably of 200 h-1 to 5000 h-1 and more preferably of 500 h-1 to 2000 h-1.

In one embodiment of the process of the invention, the molar ratio of the alkene to oxygen is from 1.0:0.1 to 2.0:1.0, preferably from 1.0:0.5 to 2.0:1.0 and more preferably from 1.0:0.8 to 2.0:1.0.

In one embodiment of the process of the invention, a first aromatic compound is formed by reaction of the alkene with the arene oxide, a second aromatic compound by reaction of the alkene with the pyridine N-oxide, and/or a third aromatic compound by reaction of the alkene with the pyrimidine N-oxide.

In one embodiment of the process of the invention, the alkene is metered into the first reactor continuously or stepwise, preferably continuously.

In one embodiment of the process of the invention, the arene oxide, pyridine N-oxide and/or pyrimidine N-oxide are metered into the first reactor continuously or stepwise, preferably continuously.

In one embodiment of the process of the invention, the alkene and the arene oxide, pyridine N-oxide and/or pyrimidine N-oxide are metered into the first reactor continuously or stepwise, preferably continuously.

In one embodiment of the process of the invention, the alkylene oxide is withdrawn from the first reactor continuously or stepwise, preferably continuously.

In one embodiment of the process of the invention, the first aromatic compound, the second aromatic compound and/or the third aromatic compound are withdrawn from the first reactor continuously or stepwise, preferably continuously.

In one embodiment of the process of the invention, the alkylene oxide and the halogenated, preferably the alkylene oxide and the first aromatic compound, the second aromatic compound and/or the third aromatic compound, are withdrawn from the first reactor continuously or stepwise, preferably continuously.

In one embodiment of the process of the invention, catalyst (A) is metered into the reactor continuously or stepwise, preferably continuously.

In one embodiment of the process of the invention, the first reactor is a stirred tank, flow tube, bubble column, loop reactor, trickle bed reactor, spray tower reactor or falling-film reactor.

In one embodiment of the process of the invention, the arene oxide, the pyridine oxide and/or the pyrimidine N-oxide is produced in a second reactor, wherein the second reactor is not the same as the first reactor.

In one embodiment of the process of the invention, the arene oxide, the pyridine N-oxide and/or the pyrimidine N-oxide is produced in the second reactor at a temperature of 20° C. to 250° C., preferably of 50° C. to 220° C. and more preferably of 100° C. to 200° C.

In one embodiment of the process of the invention, the arene oxide, the pyridine N-oxide and/or the pyrimidine N-oxide is produced in the second reactor at a pressure of 1 bara to 200 bara, preferably of 1 bara to 100 bara and more preferably of 1 bara to 60 bara.

In one embodiment of the process of the invention, the arene oxide, the pyridine N-oxide and/or the pyrimidine N-oxide is produced in the second reactor within a period of 6 min to 48 h, preferably of 6 min to 24 h and more preferably of 6 min to 3 h.

In one embodiment of the process of the invention, the first aromatic compound, the second aromatic compound and/or the third aromatic compound are metered into the second reactor continuously or stepwise, preferably continuously.

In one embodiment of the process of the invention, oxygen or the oxygen-containing gas mixture is metered into the second reactor continuously or stepwise, preferably continuously.

In one embodiment of the process of the invention, the first aromatic compound, the second aromatic compound and/or the third aromatic compound and oxygen or the oxygen-containing gas mixture are metered into the second reactor continuously or stepwise, preferably continuously.

In one embodiment of the process of the invention, the arene oxide, the pyridine N-oxide and/or the pyrimidine N-oxide are withdrawn from the second reactor continuously or stepwise, preferably continuously.

In one embodiment of the process of the invention, catalyst (C) is metered into the second reactor continuously or stepwise, preferably continuously.

In one embodiment of the process of the invention, the second reactor is a stirred tank, flow tube, bubble column, loop reactor, trickle bed reactor, spray tower reactor or falling-film reactor.

In one embodiment of the process of the invention, the arene oxide, pyridine N-oxide and/or pyrimidine N-oxide prepared in the second reactor is metered into the first reactor continuously or stepwise, preferably continuously.

The invention further provides for the use of catalyst (A) for production of the alkylene oxide of the invention by reacting the alkene of the invention with the arene oxide of the invention, the pyridine N-oxide of the invention and/or the pyrimidine N-oxide of the invention, preferably with the arene oxide of the invention and/or the pyridine N-oxide of the invention.

In a first version, the invention relates to a process for producing an alkylene oxide by reacting an alkene with an arene oxide, pyridine N-oxide and/or pyrimidine N-oxide, preferably with an arene oxide and/or pyridine N-oxide, preferably in the presence of a catalyst (A) in a first reactor, where the catalyst (A) comprises a metal (A-1) and/or a metal salt (A-2), where the metal (A-1) is copper, silver and/or gold, where the metal salt (A-2) comprises chromium (Cr), iron (Fe), cobalt (Co) and/or copper (Cu) cation(s), and where the reaction is effected in the absence of oxygen or an oxygen-containing gas mixture.

In a second version, the invention relates to a process according to the first version, wherein the alkylene oxide is one or more compound(s) and is selected from the group consisting of ethylene oxide, propylene oxide, 1,2-butylene oxide, 1,2-pentylene oxide, 1,2-hexylene oxide, 1,2-heptylene oxide and 1,2-octylene oxide, preferably ethylene oxide and propylene oxide, more preferably propylene oxide.

In a third version, the invention relates to a process according to the first or second version, wherein the arene oxide is one or more compounds of formula (I), (II), (III) and/or (IV):

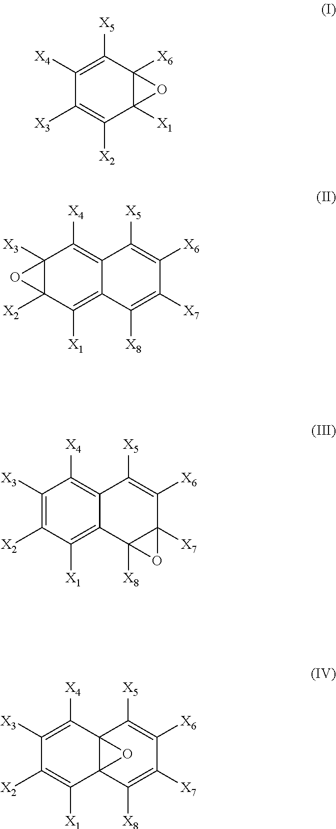

with X₁ to X₈ independently selected from the group of F, Cl, Br, CN, —CO—RCF₃, —CO—C(CH₃)₃, —CO—CH₂C(CH₃)₃, —CO—C₆H₅, —CO—OC(CH₃)₃, —CO—OCH₂C(CH₃)₃ and H, preferably F, Cl, more preferably Cl.

In a fourth version, the invention relates to a process according to the either the first or fourth embodiment, wherein the arene oxide is one or more compound(s) and is selected from the group consisting of hexafluorobenzene oxide, hexachlorobenzene oxide, 1-bromo-2,3,4-trifluorobenzene oxide, pentafluorobenzene oxide, 1,3,5-trichloro-2,4,6-trifluorobenzene oxide, 1,3,5-trifluorobenzene oxide, 1,2-dibromo-3,5-difluorobenzene oxide, 1,2,4,5-tetrafluorobenzene oxide, bromopentafluorobenzene oxide, 1,3,5-trichlorobenzene oxide, 1-bromo-3,5-dichlorobenzene oxide, orthodichlorobenzene oxide, 1,2,4,5-tetrachlorobenzene oxide, 1,2,3-trichlorobenzene oxide and 1,5-dichloro-2-fluorobenzene oxide, preferably hexafluorobenzene oxide and hexachlorobenzene oxide.

In a fifth version, the invention relates to a process according to any of the first to fourth embodiments, wherein the arene oxide is obtainable by reacting a first aromatic compound with oxygen or an oxygen-containing gas mixture in the presence of a catalyst (C).

In a sixth version, the invention relates to a process according to any of the first to fifth embodiments, wherein the arene oxide is prepared by reacting a first aromatic compound with oxygen or an oxygen-containing gas mixture in the presence of a catalyst (C).

In a seventh version, the invention relates to a process according to the fifth or sixth embodiment, wherein the first aromatic compound has a boiling temperature of 50° C. to 350° C. at 1 bara. In an eighth version, the invention relates to a process according to any of the fifth to seventh embodiments, wherein the first aromatic compound is one or more compound(s) and is selected from the group consisting of hexafluorobenzene, hexachlorobenzene, 1-bromo-2,3,4-trifluorobenzene, pentafluorobenzene, 1,3,5-trichloro-2,4,6-trifluorobenzene, 1,3,5-trifluorobenzene, 1,2-dibromo-3,5-difluorobenzene, 1,2,4,5-tetrafluorobenzene, bromopentafluorobenzene, 1,3,5-trichlorobenzene, 1-bromo-3,5-dichlorobenzene, orthodichlorobenzene, 1,2,4,5-tetrachlorobenzene, 1,2,3-trichlorobenzene and 1,5-dichloro-2-fluorobenzene, preferably hexafluorobenzene and hexachlorobenzene.

In a ninth version, the invention relates to a process according to any of the fifth to eighth embodiments, wherein the catalyst (C) is one or more compound(s) and is selected from the group consisting of silver, silver supported on magnesium silicate, and cytochrome P450 enzyme.

In a tenth version, the invention relates to a process according to any of the fifth to ninth embodiments, wherein the molar ratio of the oxygen to the first aromatic compound is from 1:500 to 1:1, preferably from 1:100 to 1:1.

In an eleventh version, the invention relates to a process according to any of the fifth to tenth embodiments, wherein the catalyst (C) is used in a calculated amount of 10 ppm to 15%, preferably of 100 ppm to 5% and more preferably of 100 ppm to 2%, based on the amount of the first aromatic compound used.

In a twelfth version, the invention relates to a process according to any of the fifth to eleventh embodiments, wherein the arene oxide is produced at a temperature of 20° C. to 250° C., preferably of 50° C. to 220° C. and more preferably of 100° C. to 200° C.

In a thirteenth version, the invention relates to a process according to any of the fifth to twelfth embodiments, wherein the arene oxide is produced at a pressure of 1 bara to 200 bara, preferably of 1 bara to 100 bara and more preferably of 1 bara to 60 bara.

In a fourteenth version, the invention relates to a process according to any of the fifth to thirteenth embodiments, wherein the arene oxide is produced within a period of 6 min to 48 h, preferably of 6 min to 24 h, and more preferably of 6 min to 3 h.

In a fifteenth version, the invention relates to a process according to any of the first to fourteenth embodiments, wherein the pyridine N-oxide is one or more compounds of formula (V):

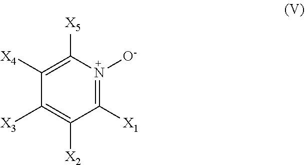

with
E selected from the group of N
X₁ to X₅ independently selected from the group of F, Cl, Br, CN, —CO—CF₃, —CO—C(CH₃)₃, —CO—CH₂C(CH₃)₃, —CO—C₆H₅, —CO—OC(CH₃)₃, —CO—OCH₂C(CH₃)₃ and H, preferably F, Cl, more preferably Cl.

In a sixteenth version, the invention relates to a process according to any of the first to fifteenth embodiments, wherein the pyridine N-oxide is one or more compound(s) and is selected from the group consisting of pentafluoropyridine N-oxide, 2-bromo-3,5-dichloropyridine N-oxide, 3-chloropyridine N-oxide, 3,6-dichloropyridine N-oxide, 3,5-dichloropyridine N-oxide, 3-chloro-2,5,6-trifluoropyridine N-oxide, 3-chloro-2,4,5,6-tetrafluoropyridine 1-N-oxide, 3-chloro-2,4,5,6-tetrafluoropyridine 3-N-oxide, preferably pentafluoropyridine N-oxide.

In a seventeenth version, the invention relates to a process according to the fifteenth or sixteenth embodiment, wherein the pyridine N-oxide is obtainable by reacting a second aromatic compound with oxygen or an oxygen-containing gas mixture in the presence of a catalyst (D).

In an eighteenth version, the invention relates to a process according to the fifteenth or sixteenth embodiment, wherein the pyridine N-oxide is prepared by reacting a second aromatic compound with oxygen or an oxygen-containing gas mixture in the presence of a catalyst (D).

In a nineteenth version, the invention relates to a process according to the seventeenth or eighteenth embodiment, wherein the second aromatic compound has a boiling temperature of 50° C. to 350° C. at 1 bara.

In a twentieth version, the invention relates to a process according to any of the seventeenth to nineteenth embodiments, wherein the second aromatic compound is one or more compound(s) and is selected from the group consisting of pentafluoropyridine N-oxide, 2-bromo-3,5-dichloropyridine, 3-chloropyridine, 3,6-dichloropyridine, 3,5-dichloropyridine, 3-chloro-2,5,6-trifluoropyridine, 3-chloro-2,4,5,6-tetrafluoropyridine, 3-chloro-2,4,5,6-tetrafluoropyridine, preferably pentafluoropyridine.

In a twenty-first version, the invention relates to a process according to any of the seventeenth to twentieth embodiments, wherein the catalyst (D) is one or more compound(s) and is selected from the group consisting of ruthenium trichloride, silver and titanium-containing zeolites.

In a twenty-second version, the invention relates to a process according to the twenty-first embodiment, wherein the catalyst (D) is applied to a catalyst support, and the catalyst support is one or more and is selected from the group consisting of magnesium silicate, silica gel, aluminum oxide, titanium dioxide and cation exchange resin.

In a twenty-third version, the invention relates to a process according to any of the seventeenth to twenty-second embodiments, wherein the molar ratio of the oxygen to the second aromatic compound is from 1:500 to 1:1, preferably from 1:100 to 1:1.

In a twenty-fourth version, the invention relates to a process according to any of the seventeenth to twenty-third, wherein the catalyst (D) is used in a calculated amount of 10 ppm to 15%, preferably of 100 ppm to 5% and more preferably of 100 ppm to 2%, based on the amount of the second aromatic compound used.

In a twenty-fifth version, the invention relates to a process according to any of the seventeenth to twenty-third, wherein the pyridine N-oxide is produced at a temperature of 0° C. to 250° C., preferably of 10° C. to 220° C. and more preferably of 20° C. to 150° C.

In a twenty-sixth version, the invention relates to a process according to any of the seventeenth to twenty-fifth, wherein the pyridine N-oxide is produced at a pressure of 1 bara to 200 bara, preferably of 1 bara to 100 bara and more preferably of 1 bara to 60 bara.

In a twenty-seventh version, the invention relates to a process according to any of the seventeenth to twenty-sixth, wherein the pyridine N-oxide is produced within a period of 6 min to 48 h, preferably of 6 min to 24 h, and more preferably of 6 min to 3 h.

In a twenty-eighth version, the invention relates to a process according to any of the first to twenty-seventh, wherein the pyrimidine N-oxide is one or more compounds of formula (VI):

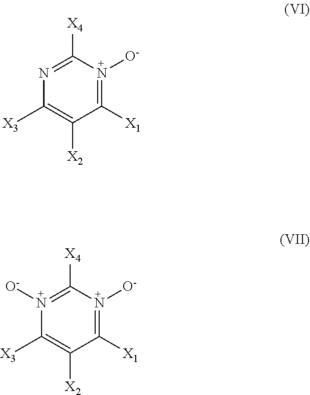

with
X₁ to X₄ independently selected from the group of F, Cl, Br, CN, —CO—CF₃, —CO—C(CH₃)₃, —CO—CH₂C(CH₃)₃, —CO—C₆H₅, —CO—OC(CH₃)₃, —CO—OCH₂C(CH₃)₃ and H, preferably F, Cl, more preferably Cl.

In a twenty-ninth version, the invention relates to a process according to any of the first to twenty-eighth embodiments, wherein the pyrimidine N-oxide is one or more compounds and is selected from the group consisting of 2-chloropyrimidine N-oxide, 2,4-dichloro-6-methylpyrimidine 1-N-oxide, 2,4-dichloro-6-methylpyrimidine 3-N-oxide, 2,5-dichloropyrimidine 1-N-oxide, 2,5-dichloropyrimidine 2-N-oxide.

In a thirtieth version, the invention relates to a process according to any of the first to twenty-ninth embodiments, wherein the pyrimidine N-oxide is obtainable by reacting a third aromatic compound with oxygen or an oxygen-containing gas mixture in the presence of a catalyst (E).

In a thirty-first version, the invention relates to a process according to any of the first to thirtieth embodiments, wherein the pyrimidine N-oxide is produced by reacting a third aromatic compound with oxygen or an oxygen-containing gas mixture in the presence of a catalyst (E).

In a thirty-second version, the invention relates to a process according to the thirtieth or thirty-first embodiment, wherein the third aromatic compound has a boiling temperature of 50° C. to 350° C. at 1 bara.

In a thirty-third version, the invention relates to a process according to any of the thirtieth to thirty-second embodiments, wherein the third aromatic compound is one or more compound(s) and is selected from the group consisting of 2-chloropyrimidine, 2,4-dichloro-6-methylpyrimidine, 2,4-dichloro-6-methylpyrimidine, 2,5-dichloropyrimidine, preferably 2,4-dichloro-6-methylpyrimidine.

In a thirty-fourth version, the invention relates to a according to any of the thirtieth to thirty-third embodiments, wherein the catalyst (E) is one or more compound(s) and is selected from the group consisting of ruthenium trichloride, titanium-containing zeolites and silver.

In a thirty-fifth version, the invention relates to a process according to any of the thirtieth to thirty-fourth embodiments, wherein the molar ratio of the oxygen to the third aromatic compound is from 1:500 to 1:1, preferably from 1:100 to 1:1.

In a thirty-sixth version, the invention relates to a process according to any of the thirtieth to thirty-fifth embodiments, wherein the catalyst (E) is used in a calculated amount of 10 ppm to 15%, preferably of 100 ppm to 5% and more preferably of 100 ppm to 2%, based on the amount of the third aromatic compound used.

In a thirty-seventh version, the invention relates to a process according to any of the thirtieth to thirty-sixth embodiments, wherein the pyrimidine N-oxide is produced at a temperature of 0° C. to 250° C., preferably of 10° C. to 220° C. and more preferably of 20° C. to 150° C.

In a thirty-eighth version, the invention relates to a process according to any of the thirtieth to thirty-seventh embodiments, wherein the pyrimidine N-oxide is produced at a pressure of 1 bara to 200 bara, preferably of 1 bara to 100 bara and more preferably of 1 bara to 60 bara.

In a thirty-ninth version, the invention relates to a process according to any of the thirtieth to thirty-eighth embodiments, wherein the pyrimidine N-oxide is produced within a period of 6 min to 48 h, preferably of 6 min to 24 h, and more preferably of 6 min to 3 h.

In a fortieth version, the invention relates to a process according to any of the first to thirty-ninth embodiments, wherein the metal (A-1) is copper (Cu), silver (Ag) and/or gold (Au), preferably silver (Ag).

In a forty-first version, the invention relates to a process according to any of the first to fortieth embodiments, wherein the metal cation of the metal salt (A-2) has an oxidation state of (+I), (+II); (+III) or (+IV), preferably of (+II); (+III) or (+IV).

In a forty-second embodiment, the invention relates to a process according to any of the first to forty-first embodiments, wherein the metal salt (A-2) is a nitrate, halide, tetrafluoroborate, paratoluenesulfonate, methanesulfonate and/or triflate, preferably a chloride.

In a forty-third version, the invention relates to a process according to any of the first to forty-second embodiments, wherein the metal salt (A-2) is one or more compound(s) and is selected from the group consisting of Cr₂(SO₄)₃, KCr(SO₄)₂, Cr(NO₃)₃, CrF₃, CrCl₃, FeCl₃, FeBr₃, iron triflate, FePO₄, Fe₂(SO4)₃, Fe(NO₃)₃, FeF₃, iron paratoluenesulfonate, CoCl₂, CoBr₂, Co(NO₃)₂, CoBr₂, CoSO₄, CoF₂, Co(BF₄)₂, Co₃(PO₄)₂, CuCl₂, CuSO₄, (CF₃SO₃)₂Cu, CuF₂, Cu(NO₃)₂, copper(II) pyrophosphate, and CuCl, CuI, CuBr, preferably CrCl₃, FeCl₃, CoCl₂ and CuCl₂.

In a forty-fourth version, the invention relates to a process according to any of the first to forty-third embodiments, wherein catalyst (A) is used in a calculated amount of 10 ppm to 15%, preferably of 100 ppm to 5% and more preferably of 100 ppm to 2%, based on the mass of all components used.

In a forty-fifth version, the invention relates to a process according to any of the first to forty-fourth embodiments, wherein the catalyst (A) is applied to a catalyst support (B) to form a supported catalyst (A′).

In a forty-sixth embodiment, the invention relates to a process according to the forty-fifth embodiment, wherein the catalyst support (B) is a metal oxide, an alkaline earth metal carbonate, a silicate, a silicon carbide, a silicon oxycarbide, a silicon nitride, a silicon oxynitride and/or a silicon dioxide.

In a forty-seventh version, the invention relates to a process according to the forty-fifth or forty-sixth embodiment, wherein the catalyst support (B) is one or more compound(s) and is selected from the group consisting of aluminum oxide aluminum dioxide, silica, titanium dioxide, zirconium dioxide, aluminium carbonate, phyllosilicate, such as talc, kaolinite and pyrophyllite, and titanium dioxide.

In a forty-eighth embodiment, the invention relates to a process according to any of the forty-fifth to forty-seventh embodiments, wherein the catalyst (A) is applied to the catalyst support (B) in a calculated proportion by mass of 1.0% by weight to 30.0% by weight.

In a forty-ninth embodiment, the invention relates to a process according to any of the forty-fifth to forty-eighth embodiments, wherein the catalyst (A) is applied to the catalyst support (B) by the wet infiltration method or the incipient wetness method to form the supported catalyst (A′).

In a fiftieth version, the invention relates to a process according to any of the first to forty-ninth embodiments, wherein the molar ratio of the alkene to the arene oxide is from 1:0.01 to 10:1, preferably from 1:0.1 to 1:1.

In a fifty-first version, the invention relates to a process according to any of the first to fiftieth embodiments, wherein the alkylene oxide is produced in the presence of a solvent.

In a fifty-second version, the invention relates to a process according to the fifty-first embodiment, wherein the solvent is one or more compound(s) and is selected from the group consisting of CO2, water, perfluoromethyldecalin, perfluorodecalin, perfluoroperhydrophenanthrene, perfluoro(butyltetrahydrofuran), tetrahydrofuran, 2-methyl-THF, acetic acid, acetonitrile, dimethyl sulfoxide, sulfolane, acetone, ethyl methyl ketone, dimethylformamide, dichloromethane, chloroform, tetrachloromethane, N-methyl-2-pyrrolidinone, methyl t-butyl ether (MTBE), dimethyl sulfoxide (DMSO), hexamethylphosphoramide, dichlorobenzene, 1,2-dichloroethylene, 1,1,1,3,3,3-hexafluoroisopropanol, perfluoro-tert-butyl alcohol, 1,1,2,3,3-pentafluoropropane, 1-bromo-2-chloro-1,1,2-trifluoroethane, 1,2-dichloro-1,1,2,3,3,3-hexafluoropropane, ethylene glycol, glycerol, and phenol.

In a fifty-third version, the invention relates to a process according to the fifty-first or fifty-second embodiment, wherein the production is effected at a temperature of 20° C. to 200° C., preferably of 50° C. to 160° C. and more preferably of 100° C. to 150° C.

In a fifty-fourth version, the invention relates to a process according to any of the fifty-first to fifty-third embodiments, wherein the production is effected at a pressure of 1 bara to 200 bara, preferably of 1 bara to 35 bara and more preferably of 1 bara to 28 bara.

In a fifty-fifth version, the invention relates to a process according to any of the fifty-first to fifty-fourth embodiments, wherein the production is effected within a period of 6 min to 48 h, preferably of 6 min to 24 h, and more preferably of 6 min to 3 h.

In a fifty-sixth version, the invention relates to a process according to any of the fifty-first to fifty-fifth embodiments, wherein the molar ratio of the alkene to oxygen is from 1:100 to 100:1, preferably from 1:30 to 30:1.

In a fifty-seventh version, the invention relates to a process according to any of the first to fifty-sixth embodiments, wherein the production is effected in the absence of a solvent.

In a fifty-eighth version, the invention relates to a process according to the fifty-seventh embodiment, wherein the production using supported catalyst (A′) according to any of the forty-fifth to forty-ninth embodiments.

In a fifty-ninth version, the invention relates to a process according to the fifty-seventh or fifty-eighth embodiment, wherein the production is effected at a temperature of 20° C. to 500° C., preferably of 50° C. to 400° C. and more preferably of 50° C. to 250° C.

In a sixtieth version, the invention relates to a process according to any of the fifty-seventh to fifty-ninth embodiments, wherein the production is effected at a pressure of 1 bara to 200 bara, preferably of 2 bara to 100 bara and more preferably of 2 bara to 50 bara.

In a sixty-first version, the invention relates to a process according to any of the fifty-seventh to sixtieth embodiments, wherein the production is effected at a space velocity of 100 h 1 to 10 000 h 1, preferably of 200 h 1 to 5000 h 1 and more preferably of 500 h 1 to 2000 h 1.

In a sixty-second version, the invention relates to a process according to any of the fifty-seventh to sixty-first embodiments, wherein the molar ratio of the alkene to oxygen is from 1.0:0.1 to 2.0:1.0, preferably from 1.0:0.5 to 2.0:1.0 and more preferably from 1.0:0.8 to 2.0:1.0.

In a sixty-third version, the invention relates to a process according to any of the first to sixty-second embodiments, wherein a first aromatic compound is formed by reaction of the alkene with the arene oxide, a second aromatic compound by reaction of the alkene with the pyridine N-oxide, and/or a third aromatic compound by reaction of the alkene with the pyrimidine N-oxide.

In a sixty-fourth version, the invention relates to a process according to any of the first to sixty-third embodiments, wherein the alkene is metered into the first reactor continuously or stepwise, preferably continuously.

In a sixty-fifth version, the invention relates to a process according to any of the first to sixty-fourth embodiments, wherein the arene oxide, pyridine N-oxide and/or pyrimidine N-oxide is metered into the first reactor continuously or stepwise, preferably continuously.

In a sixty-sixth version, the invention relates to a process according to any of the first to sixty-fifth embodiments, wherein the alkene and the arene oxide, pyridine N-oxide and/or pyrimidine N-oxide are metered into the first reactor continuously or stepwise, preferably continuously.

In a sixty-seventh version, the invention relates to a process according to any of the first to sixty-sixth embodiments, wherein the alkylene oxide is withdrawn from the first reactor continuously or stepwise, preferably continuously.

In a sixty-eighth version, the invention relates to a process according to any of the sixty-third to sixty-seventh embodiments, wherein the first aromatic compound, the second aromatic compound and/or the third aromatic compound is withdrawn from the first reactor continuously or stepwise, preferably continuously.

In a sixty-ninth version, the invention relates to a process according to any of the sixty-third to sixty-eighth embodiments, wherein the alkylene oxide and the halogenated, preferably the alkylene oxide and the first aromatic compound, the second aromatic compound and/or the third aromatic compound, are withdrawn from the first reactor continuously or stepwise, preferably continuously.

In a seventieth version, the invention relates to a process according to any of the first to sixty-ninth embodiments, wherein the catalyst (A) is metered into the reactor continuously or stepwise, preferably continuously.

In a seventy-first version, the invention relates to a process according to any of the first to seventieth embodiments, wherein the first reactor is a stirred tank, flow tube, bubble column, loop reactor, trickle bed reactor, spray tower reactor or falling-film reactor.

In a seventy-second version, the invention relates to a process according to any of the first to seventy-first embodiments, wherein the arene oxide, the pyridine oxide and/or the pyrimidine N-oxide is produced in a second reactor, wherein the second reactor is not the same as the first reactor.

In a seventy-third version, the invention relates to a process according to the seventy-second embodiment, wherein the arene oxide, the pyridine N-oxide and/or the pyrimidine N-oxide is produced at a temperature of 20° C. to 250° C., preferably of 50° C. to 220° C. and more preferably of 100° C. to 200° C.

In a seventy-fourth version, the invention relates to a process according to the seventy-second or seventy-third embodiment, wherein the pyridine N-oxide and/or the pyrimidine N-oxide is produced at a pressure of 1 bara to 200 bara, preferably of 1 bara to 100 bara and more preferably of 1 bara to 60 bara.

In a seventy-fifth version, the invention relates to a process according to any of the seventy-second to seventy-fourth embodiments, wherein the arene oxide, the pyridine N-oxide and/or the pyrimidine N-oxide is produced in the second reactor within a period of 6 min to 48 h, preferably of 6 min to 24 h, and more preferably of 6 min to 3 h.

In a seventy-sixth version, the invention relates to a process according to any of the seventy-second to seventy-fifth embodiments, wherein the first aromatic compound, the second aromatic compound and/or the third aromatic compound is metered into the second reactor continuously or stepwise, preferably continuously.

In a seventy-seventh version, the invention relates to a process according to any of the seventy-second to seventy-sixth embodiments, wherein oxygen or the oxygen-containing gas mixture is metered into the second reactor continuously or stepwise, preferably continuously.

In a seventy-eighth version, the invention relates to a process according to any of the seventy-second to seventy-seventh embodiments, wherein the first aromatic compound, the second aromatic compound and/or the third aromatic compound and oxygen or the oxygen-containing gas mixture are metered into the second reactor continuously or stepwise, preferably continuously.

In a seventy-ninth version, the invention relates to a process according to any of the seventy-second to seventy-eighth embodiments, wherein the arene oxide, the pyridine N-oxide and/or the pyrimidine N-oxide is withdrawn from the second reactor continuously or stepwise, preferably continuously.

In an eightieth version, the invention relates to a process according to any of the seventy-second to seventy-ninth embodiments, wherein the catalyst (C) is metered into the reactor continuously or stepwise, preferably continuously.

In an eighty-first version, the invention relates to a process according to any of the seventy-second to eightieth embodiments, wherein the second reactor is a stirred tank, flow tube, bubble column, loop reactor, trickle bed reactor, spray tower reactor or falling-film reactor.

In an eighty-second version, the invention relates to a process according to any of the seventy-second to eight-first embodiments, wherein the arene oxide, pyridine N-oxide and/or pyrimidine N-oxide produced in the second reactor is metered into the first reactor continuously or stepwise, preferably continuously.

In an eighty-third version, the invention relates to a process according to any of the first to eighty-second embodiments, wherein the alkene is one or more compound(s) and is selected from the group consisting of ethene, propene, butene, 1-octene, butadiene, butane-1,4-diol diallyl ether, allyl chloride, allyl alcohol, styrene, cyclopentene, cyclohexene, phenyl allyl ether, diallyl ether, n-butyl allyl ether, tert-butyl allyl ether, bisphenol A diallyl ether, resorcinol diallyl ether, triphenylolmethane triallyl ether, cyclohexane-1,2-dicarboxylic acid bis(allyl ester), isocyanuric acid tris(prop-2,3-ene) ester and mixtures of these alkenes, preferably ethene, propene and allyl chloride, and more preferably propene.

### EXAMPLES

### Chemicals Used

copper, powder, 99.999%, Sigma Aldrich
copper monochloride, ≥99.995%, Sigma Aldrich
silver, powder, 2-3.5 μm, ≥99.9%, Sigma Aldrich
iron(III) chloride, ≥99.99%, Sigma Aldrich
chromium(III) chloride, 99.99%, Sigma Aldrich
ruthenium(III) chloride hydrate, 99.98%, Sigma Aldrich

All chemicals were used as obtained.

### Gas Chromatography Analysis

The gas chromatography analysis (GC for short) of liquid and gas samples was performed in accordance with “Determine Impurities in High-Purity Propylene Oxide with Agilent J&W PoraBOND U” by Dianli Ma, Ningbo ZRCC Lyondell Chemical Co., Ltd Zhejiang, China, and Yun Zou, Hua Wu, Agilent Technologies, Inc. On the basis of GC, conversions of propene, yields of propylene oxides and selectivities were ascertained.

### Simulation Method

All quantum mechanical calculations were carried out using the software package TURBOMOLE version 7.4.1 from Cosmologic GmbH & Co. KG. The density functional theory method used was the TPSS density functional, implemented as unrestricted DFT for spin contamination of open-shell systems, with a def2-SVP basis set, as implemented as standard in the Turbomole software package. The energies obtained were refined by the DFT method described and with a basis set of def2-TZVP quality.

### FIG. 1: Input Geometry for the Quantum Chemical Calculations of the Transition States of the Catalyzed Oxygen Transfer

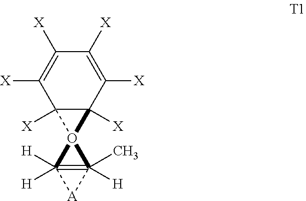

Transition states were calculated by gradient-based Monte Carlo, as described in application WO 2020/079094 A2. For this purpose, a structure was drawn according to transition state T1 (FIG. 1). The bonds drawn in bold were set at an atomic distance of 1.90 Å (190 μm), and the structure thus obtained was converted to Cartesian coordinates. The atomic indices in the set of Cartesian coordinates of the bonds shown in bold in FIG. 1 were set as function space in the gradient-based Monte Carlo program, and the Monte Carlo procedure was executed until the corresponding transition states T1 were obtained. Thereafter, the Cartesian coordinates of structures T1 that were obtained in this way were manipulated in order to obtain the corresponding reactant catalyst complexes or the corresponding catalyst product complexes. For this purpose, the bond drawn in bold (FIG. 1) between oxygen and the aromatic carbon atom of the haloaromatic was i) extended and ii) shortened by 0.20 Å (20 μm). The structures i) and ii) thus obtained were expressed in the form of Cartesian coordinates and subjected to optimization of geometry by the DFT method described, and the resultant geometries were used for the calculation of the activation energies.

Oxidation mediators identified as being suitable for the selective oxidation of propene to propylene oxide, by means of quantum-chemical simulations (working examples), were arene oxides of hexafluorobenzene and hexachlorobenzene, and pentafluoropyridine N-oxide. Hexafluorobenzene and hexachlorobenzene can be readily converted to the respective arene oxides, are unlikely to undergo unwanted breakdown reactions, and transfer the atomic oxygen selectively to the propene with exclusive PO formation (cf. FIG. 2). The same is true of pentafluoropyridine N-oxide. Arene oxides of various halogenated benzenes are also commercially available.

By way of experimental evidence of utility of such arene oxides and of structurally related pyridine N-oxides for formation of alkylene oxides, quantum-chemical simulations were conducted, in which the transfer of the bound oxygen to the propene was examined. In the case of the arene oxides, the bound oxygen atom is transferred to the double bond of propene, with rearomatization of the aromatic hydrocarbon. In the case of pentafluoropyridine N-oxide, the electronically uncharged pyridine bond system is formed. It is the reformation of the aromatic systems that acts as the driving force for transfer of the oxygen to the propene. In both compound classes, there is comprehensive and selective recovery of the starting components resulting from the oxygen transfer. Recovery and reoxidation to form the oxidation mediator structure is therefore always possible. The starting compounds of the oxidation mediators are recycled.

FIG. 2: Reaction Sequence of the Catalytic Formation of the Arene Oxide in the Presence of Catalyst (D), Followed by the Oxidation of Propene with an Arene Oxide Using Catalyst (A)

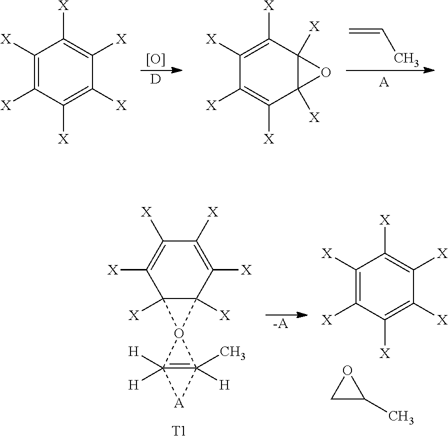

TABLE 1
Overview of simulation-calculated activation energies of the transfer
of oxygen to propene with various oxidation mediators

| Oxidation mediator oxide | Ea, simulated [kcal/mol] |
|---|---|
| Hexafluorobenzene oxide | 27.3 |
| Hexachlorobenzene oxide | 21.6 |
| Pentafluoropyridine N-oxide | 32.5 |

The values shown in table 2 suggest that, in the case of hexachlorobenzene oxide, even without further auxiliaries such as particular catalysts, alkoxylation of propene (epoxidation of propene, propoxylation) can be achieved solely through appropriate reaction temperatures. For hexafluorobenzene oxide and pentafluoropyridine N-oxide, by contrast, the activation energies calculated indicate the additional use of a suitable catalyst in order to enable the epoxidation of propene.

Suitable catalysts (A) identified by quantum-chemical simulations (working examples) are metallic silver, metallic copper, copper(I) chloride, iron(III) chloride and chromium(III) chloride.

TABLE 2
Overview of simulation-calculated activation energies
of the transfer of oxygen from hexafluorobenzene
oxide to propene with various catalysts (A)

| Catalyst (A) | Ea, simulated [kcal/mol] |
|---|---|
| Uncatalyzed, hexafluorobenzene oxide | 27.3 |
| Ag(0) | 17.4 |
| Cu(0) | 18.2 |
| NiCl₂En₂ (comp.) | 28.0 |
| Fe(III)Cl₃ | 14.3 |
| Cr(III)Cl₃ | 17.3 |

The calculated values in table 2 show that the activation energy of the uncatalyzed reaction of 27.3 kcal/mol can be distinctly reduced by the catalysts (A) used. Moreover, the simulated results illustrate that the activation energies are distinctly dependent on the respective selection of the catalyst (A). In the case of hexachlorobenzene oxide, the calculated activation energy of 21.6 kcal/mol is advantageous even without the use of a catalyst (A).

TABLE 3
Overview of simulation-calculated activation energies of the transfer
of oxygen from pentafluoropyridine N-oxide to propene

| | Ea, simulated | Ea, simulated, |
|---|---|---|
| | (propoxylation) | side reaction |
| Catalyst (A) | [kcal/mol] | [kcal/mol] |
| Uncatalyzed (comp.) | 32.5 | |
| Ag(0) | 15.2 | 22.7 |
| Au(0) | 11.2 | 20.2 |
| Cu(0) | 10.3 | 19.9 |
| Cu(II)Cl₂ | 14.0 | 33.7 |
| Cr(III)Cl₃ | 15.9 | 19.6 |
| Co(0) (comp.) | 26.3 | 30.1 |
| Co(II)Cl₂ | 20.5 | 30.3 |
| Ru(III)Cl₃ (comp.) | —¹⁾ | 25.3 |
| Ru(II)Cl₂ (comp.) | —¹⁾ | — |
| [Pt(II)Cl₃]⁻ (comp.) | 41.6 | — |

¹⁾Rather than leading to propylene oxide or corresponding intermediates, leads further to C—C surgeon of propylene (bond distance on the product side in the simulations is 3.112 Å to 4.124 Å, while the calculated C—C bond distance in the case of propylene oxide is 1.475 Å).

Table 3 shows the simulated activation energies for the epoxidation of propene with hexafluoropyridine N-oxide with and without catalysts. The calculated values show that the activation energy of the uncatalyzed reaction of 32.5 kcal/mol can be reduced, in some cases distinctly reduced, by the catalysts A used (cf. “Ea, simulated (propoxylation)” column).

1) Alkoxylation of Propene with Hexachlorobenzene Oxide:

Hexachlorobenzene oxide was obtained according to the prior art by catalytic oxidation of hexachlorobenzene. A 1 M solution of hexachlorobenzene in perfluorodecalin was produced. Then a pressure-resistant 1 l reactor with stirrer system, pressure relief valve, pressure sensor, riser tube for removal of liquid, and sparging and degassing conduits was initially charged with 200 ml of perfluorodecalin under inert conditions. 0.400 mol of propene was then injected into the reaction vessel (about 11 bar). The internal reactor temperature was set at 135° C. by closed-loop control, and 0.400 mol of hexachlorobenzene oxide was added in the form of the previously prepared perfluorodecalin solution. The progress and endpoint of the reaction were ascertained from the pressure profile and by withdrawal of liquid and gas samples and analysis thereof by GC analyzed. After the reaction had ended, the reactor was cooled down to 40° C., then decompressed, and the propylene oxide reaction product was distilled off into a cooled receiver via the degassing conduit. The theoretical yield of propylene oxide was 23.2 g.

2) Alkoxylation of Propene with Hexachlorobenzene Oxide:

A pressure-resistant 1 l reactor with stirrer system, pressure relief valve, pressure sensor, riser tube for removal of liquid, and sparging and degassing conduits was initially charged with 200 ml of perfluorodecalin, 0.400 mol of hexachlorobenzene, 0.040 mol of silver (powder, 2-3.5 μm) under inert conditions, and stirred vigorously. 0.400 mol of oxygen was then injected into the reaction vessel. The internal reactor temperature was set at 200° C. by closed-loop control, and the pressure profile was recorded until constant. This was followed by cooling to 135° C., inertization with nitrogen and addition of 0.400 mol of propene. The progress and endpoint of the reaction were ascertained from the pressure profile and by withdrawal of liquid and gas samples and analysis thereof by GC analyzed. After the reaction had ended, the reactor was cooled down to 40° C., then decompressed, and the propylene oxide reaction product was distilled off into a cooled receiver via the degassing conduit.

The theoretical yield of propylene oxide was 23.2 g.

3) Alkoxylation of Propene with Hexafluorobenzene Oxide:

Hexafluorobenzene oxide was obtained by partial oxidation of hexafluorobenzene with oxygen in a pressure reactor. The conversion of hexafluorobenzene was chosen such that the concentration of hexafluorobenzene oxide was 1 mol/l (1 M). Then a pressure-resistant 1 l reactor with stirrer system, pressure relief valve, pressure sensor, riser tube for removal of liquid, and sparging and degassing conduits was initially charged with 200 ml of hexafluorobenzene and, for each experiment, 0.020 mol of the respective catalyst identified as working example in table 2, under inert conditions. 0.400 mol of propene was then injected into the reaction vessel (about 11 bar). The internal reactor temperature was set at 60° C. by closed-loop control, and 0.400 mol of hexafluorobenzene oxide was added in the form of the previously prepared solution. The progress and endpoint of the reaction were ascertained from the pressure profile and by withdrawal of liquid and gas samples and analysis thereof by GC analyzed. After the reaction had ended, the reactor was cooled down to 40° C., then decompressed, and the propylene oxide reaction product was distilled off into a cooled receiver via the degassing conduit.

The theoretical yield of propylene oxide in all five experiments was 23.2 g.

4) Alkoxylation of Propene with Hexafluorobenzene Oxide:

The continuous epoxidation of propene with hexafluorobenzene oxide was conducted analogously to experimental method 3. However, the hexafluorobenzene oxide and the propene were metered continuously into the reactor (molar ratio 1.05:1), and the reaction mixture was withdrawn volumetrically in the same way and worked up by distillation. The mass and volume flows were set by closed-loop control so as to result in a dwell time of 90 min. After 6 hours, the reaction was in a steady state. Conversions and selectivities, analogously to experiment 3, were observed in all experiments with the different catalysts of the invention from table 2.

4) Alkoxylation of Propene with Hexafluorobenzene Oxide:

The continuous epoxidation of gaseous propene with gaseous hexafluorobenzene oxide was conducted analogously to experimental method 4. However, the hexafluorobenzene oxide and the propene were evaporated and introduced continuously into an inertized pressure-rated and pressure-safeguarded flow reactor that contained silver (powder, 2-3.5 μm) as solid catalyst. The molar ratio was 5:1 hexafluorobenzene oxide to propene, and a reaction temperature of 120° C. was set by closed-loop control. Partial conversions of propene and propylene oxide selectivities analogous to experiments 3 and 4 were observed. There was no exact determination of dwell times.

5) Epoxidation of Propene with Pentafluoropyridine N-Oxide:

Pentafluoropyridine N-oxide was prepared analogously to the manner described in the prior art for N-oxides (Chem. Commun. 2002, 1040-1041) and provided as a 1 M dichloroethane solution. Then a pressure-resistant 1 l reactor with stirrer system, pressure relief valve, pressure sensor, riser tube for removal of liquid, and sparging and degassing conduits was initially charged with 200 ml of dichloroethane and, for each experiment, 0.020 mol of the respective catalyst identified as working example in table 3, under inert conditions. 0.400 mol of propene was then injected into the reaction vessel (about 11 bar). The internal reactor temperature was set at 60° C. by closed-loop control, and 0.400 mol of pentafluoropyridine N-oxide was added in the form of the previously prepared solution. The progress and endpoint of the reaction were ascertained from the pressure profile and by withdrawal of liquid and gas samples and analysis thereof by GC analyzed. After the reaction had ended, the reactor was cooled down to 40° C., then decompressed, and the propylene oxide reaction product was distilled off into a cooled receiver via the degassing conduit.

The theoretical yield of propylene oxide in all five experiments is 23.2 g.

1. A process for producing an alkylene oxide comprising reacting an alkene with an arene oxide, pyridine N-oxide and/or pyrimidine N-oxide in the presence of a catalyst (A) in a first reactor, wherein the catalyst (A) comprises a metal (A-1) and/or a metal salt (A-2),
wherein the metal (A-1) comprises copper, silver and/or gold,
wherein the metal salt (A-2) comprises chromium (Cr), iron (Fe), cobalt (Co) and/or copper (Cu) cation(s),
and wherein the reaction is effected in the absence of oxygen or an oxygen-containing gas mixture.

2. The process as claimed in claim 1, wherein the alkylene oxide is one or more compound(s) and comprises ethylene oxide, propylene oxide, 1,2-butylene oxide, 1,2-pentylene oxide, 1,2-hexylene oxide, 1,2-heptylene oxide, 1,2-octylene oxide, or a mixture thereof.

3. The process as claimed in claim 1, wherein the alkene is one or more compound(s) and comprises ethene, propene, butene, 1-octene, butadiene, butane-1,4-diol diallyl ether, allyl chloride, allyl alcohol, styrene, cyclopentene, cyclohexene, phenyl allyl ether, diallyl ether, n-butyl allyl ether, tert-butyl allyl ether, bisphenol A diallyl ether, resorcinol diallyl ether, triphenylolmethane triallyl ether, cyclohexane-1,2-dicarboxylic acid bis(allyl ester), isocyanuric acid tris(prop-2,3-ene) ester, or a mixture thereof.

4. The process as claimed in claim 1, wherein the arene oxide is one or more compound(s) and comprises hexafluorobenzene oxide, hexachlorobenzene oxide, 1-bromo-2,3,4-trifluorobenzene oxide, pentafluorobenzene oxide, 1,3,5-trichloro-2,4,6-trifluorobenzene oxide, 1,3,5-trifluorobenzene oxide, 1,2-dibromo-3,5-difluorobenzene oxide, 1,2,4,5-tetrafluorobenzene oxide, bromopentafluorobenzene oxide, 1,3,5-trichlorobenzene oxide, 1-bromo-3,5-dichlorobenzene oxide, orthodichlorobenzene oxide, 1,2,4,5-tetrachlorobenzene oxide, 1,2,3-trichlorobenzene oxide and 1,5-dichloro-2-fluorobenzene oxide, preferably hexafluorobenzene oxide, hexachlorobenzene oxide, or a mixture thereof.

5. The process as claimed in claim 1, wherein the pyridine N-oxide is one or more compound(s) and comprises pentafluoropyridine N-oxide, 2-bromo-3,5-dichloropyridine N-oxide, 3-chloropyridine N-oxide, 3,6-dichloropyridine N-oxide, 3,5-dichloropyridine N-oxide, 3-chloro-2,5,6-trifluoropyridine N-oxide, 3-chloro-2,4,5,6-tetrafluoropyridine 1-N-oxide, 3-chloro-2,4,5,6-tetrafluoropyridine 3-N-oxide, or a mixture thereof.

6. The process as claimed in claim 1, wherein the pyrimidine N-oxide is one or more compounds and comprises 2-chloropyrimidine N-oxide, 2,4-dichloro-6-methylpyrimidine 1-N-oxide, 2,4-dichloro-6-methylpyrimidine 3-N-oxide, 2,5-dichloropyrimidine 1-N-oxide, 2,5-dichloropyrimidine 2-N-oxide, or a mixture thereof.

7. The process as claimed in claim 1, wherein the metal comprises silver (Ag).

8. The process as claimed in claim 1, wherein the metal cation of the metal salt (A-2) has an oxidation state of (+I), (+II); (+III) or (+IV).

9. The process as claimed in claim 1, wherein the metal salt (A-2) is one or more compound(s) and comprises Cr₂(SO₄)₃, KCr(SO₄)₂, Cr(NO₃)₃, CrF₃, CrCl₃, FeCl₃, FeBr₃, iron triflate, FePO₄, Fe₂(SO₄)₃, Fe(NO₃)₃, FeF₃, iron paratoluenesulfonate, CoCl₂, CoBr₂, Co(NO₃)₂, CoBr₂, CoSO₄, CoF₂, Co(BF₄)₂, CO₃(PO₄)₂, CuCl₂, CuSO₄, (CF₃SO₃)₂Cu, CuF₂, Cu(NO₃)₂, copper(II) pyrophosphate, CuCl, CuI, CuBr, or a mixture thereof.

10. The process as claimed in claim 1, wherein the production of the alkylene oxide is effected in the presence of a solvent.

11. The process as claimed in claim 1, wherein the production of the alkylene oxide is effected in the absence of a solvent.

12. The process as claimed in claim 1, wherein the alkene and the arene oxide, pyridine N-oxide and/or pyrimidine N-oxide are metered into the first reactor continuously or stepwise.

13. The process as claimed in claim 1, wherein the alkylene oxide is withdrawn from the first reactor continuously or stepwise.

14. The process as claimed in claim 1, wherein the arene oxide, the pyridine oxide and/or the pyrimidine N-oxide is produced in a second reactor, wherein the second reactor is not the same as the first reactor.

15. The process as claimed in claim 14, wherein the arene oxide, pyridine N-oxide and/or pyrimidine N-oxide produced in the second reactor is metered into the first reactor continuously or stepwise.
